SEQUENCE LISTING (4 sequences)

SEQ ID NO: 1 - DNA, 19 bp, Artificial
  synthetic
atgaccccaatacgcaaaa

SEQ ID NO: 2 - DNA, 20 bp, Artificial
  synthetic
cgaagtttcatcatgcggag

SEQ ID NO: 3 - DNA, 20 bp, Artificial
  synthetic
atgacccaccaatcacatgc

SEQ ID NO: 4 - DNA, 20 bp, Artificial
  synthetic
atcacatggctaggccggag

(19) United States
(12) Patent Application Publication
(10) Pub. No.: US 2024/0052352 A1
(43) Pub. Date: Feb. 15, 2024

(54) COMPOSITIONS AND METHODS FOR CHARACTERIZING AND TREATING DISEASES AND DISORDERS ASSOCIATED WITH MULTIPLE ORGAN FAILURE

(71) Applicant: Arizona Board of Regents on Behalf of the University of Arizona, Tucson, AZ (US)

(72) Inventor: Floyd H. Chilton, Tucson, AZ (US)

(21) Appl. No.: 18/278,196

(22) PCT Filed: Feb. 22, 2022
(86) PCT No.: PCT/US2022/017274

§ 371 Date: Aug. 22, 2023

Related U.S. Application Data
(60) Provisional application No. 63/151,858, filed on Feb. 22, 2021.

(51) Int. Cl. C12N 15/113; C12N 9/20

(52) U.S. Cl. CPC C12N 15/1137; C12N 9/20; C12Y 301/01004; C12N 2310/11; C12N 2310/14

(57) ABSTRACT

Provided herein are biomarkers for screening and monitoring of conditions, diseases, and disorders. In particular, provided herein are sPLA2 biomarkers for use in characterizing, prognosing, and treating disorders associated with elevated sPLA2.

### STATEMENT REGARDING FEDERALLY SPONSORED RESEARCH OR DEVELOPMENT

This invention was made with government support under Grant No. AT008621 awarded by National Institutes of Health. The government has certain rights in the invention.

### FIELD OF THE INVENTION

Provided herein are biomarkers for screening and monitoring of conditions, diseases, and disorders. In particular, provided herein are secreted PLA2 (e.g., sPLA2-IIA) and associated biomarkers for use in characterizing, prognosing, and treating disorders associated with elevated sPLA2.

### BACKGROUND

COVID-19 is a respiratory condition caused by the SARS-CoV-2 coronavirus. Some people are infected but don't notice any symptoms. Most people will have mild symptoms and get better on their own. But about 1 in 6 will have severe problems, such as trouble breathing. The odds of more serious symptoms are higher for older patients or those with another health condition like diabetes or heart disease.

Common symptoms include fever, fatigue, a dry cough, loss of appetite, body aches, shortness of breath, and mucus or phlegm. COVID-19 is typically diagnosed via a PCR test on a nasal or throat swab.

Severity is initially classified by respiratory factors such as respiration rate, oxygen saturation and pulmonary lesion progression and more critical cases are often complicated by organ dysfunction, including septic shock, heart failure and disseminated intravascular coagulation.

Most people diagnosed with COVID-19 do not require medical treatment. Severe cases are treated with oxygen nasal cannulas or a ventilator.

Drugs used to treat COVID-19 include the anti-viral drug remdesivir, blood thinners such as heparin, monoclonal antibodies including casirivimab and imdevimab, convalescent plasma, and dexamethasone. There is no specific therapy for severe COVID-19.

As new highly transmissible and potential vaccine-evading SARS-CoV-2 strains emerge with increasing disease-associated mortality and morbidity, there is an urgent need to elucidate central molecular and cellular mechanisms that limit host fitness in severe and lethal COVID-19 cases.

Infection with SARS-CoV-2 can also lead to lingering or new symptoms beyond the acute COVID-19 illness, currently termed post-acute sequelae of SARS-CoV-2 infection (PASC). In fact, while the exact epidemiology is unknown, as most reports are limited by selection bias and follow up, early reports of prolonged or new symptoms that develop after acute COVID-19 are staggering. Current studies report a high percentage of patients reporting PASC symptoms for weeks to months after the acute infection. In one large international study, 91% of participants reported symptoms longer than 35 weeks. Fatigue/malaise and dyspnea are two of the most common and debilitating symptoms reported, with either still reported in >50% and >37%, respectively at 12 months.

Unfortunately, despite a high percentage of persistently reported dyspnea and fatigue, there are inconsistent associations between objective clinical, laboratory, or radiographic findings and severity of symptoms. As such, determining underlying pathobiological mechanisms to identify potential therapeutic targets are desperately needed.

### SUMMARY

The present disclosure addresses the need for targeted therapy for diseases and disorders such as COVID-19, other disorders associated with multiple organ failure, and prolonged or new symptoms that develop after acute COVID-19 (such as PASC). By identifying individuals with high levels of isoforms of secreted PLA2 (sPLA2) and blood urea nitrogen (BUN) alone or in combination and providing targeted treatment, the present disclosure provides improved care for such patients.

For example, in some embodiments, provided herein is a method of treating a condition, disease or disorder in a subject, comprising: a) assaying a sample from said subject for the level of any low MW, Ca⁺⁺ dependent secreted phospholipase A2 (such as sPLA2-IIA or other secreted isoforms (e.g., PLA2G12B, PLA2G1B, PLA2G16, PLA2G5, PLA2G10, PLA2G2C, PLA2G2E, PLA2G7 or PLA2G2D)); and b) administering an sPLA2 (e.g., sPLA2-IIA) inhibitor to the subject when the sample has an elevated level of sPLA2. In some embodiments, the patient also has a blood urea nitrogen (BUN) level greater than or equal to 16 mg/dl. In some embodiments, the sPLA2 is catalytically active. In some embodiments, the sample is blood or a blood product (e.g., plasma). In some embodiments, the method further comprises assaying one or more of the subject's respiration rate, oxygen saturation or pulmonary lesion progression.

The present disclosure is not limited to a particular method of assaying the level of sPLA2 (e.g., sPLA2-IIA). Examples include, but are not limited to, immunoassays (e.g., ELISA assays, fluorescent immunoassay, chemiluminescent immunoassay, radioimmunoassay, colorimetric enzyme activity assay, and fluorometric enzyme activity assay.

Any suitable sPLA2 (e.g., sPLA2-IIA) inhibitor may be utilized in the methods described herein. Examples include but are not limited to, antibodies, nucleic acids (e.g., antisense nucleic acids, siRNAs, shRNAs, miRNAs, etc.), and small molecules (e.g., varespladib methyl, AZD2716, 7,7-Dimethyleicosadienoic Acid (DEDA), oleyloxyethyl phosphorylcholine, luffariellolide, thioetheramide PC, 4-[(1-oxo-7-phenylheptyl)amino]-(4R)-octanoic acid, LY315920, or γS-[(1-oxo-7-phenylheptyl)amino]-4-(phenylmethoxy)-benzenepentanoic acid).

The present disclosure is not limited to particular conditions, diseases, or disorders. Examples include but are not limited to, a respiratory disorder, a trauma, a bacterial infection, septic shock, heart failure, disseminated intravascular coagulation or prolonged or new symptoms that develop after an infection such as acute COVID-19. In some embodiments, the respiratory disorder is or includes acute respiratory distress syndrome (ARDS). In some embodiments, the subject is infected with or has been infected with the SARS-CoV-2 virus. In some embodiments, the subject has one or more symptoms of COVID-19. In some embodiments, the subject is at increased risk of severe disease or death from the respiratory disorder (e.g., due to age over 65 or comorbidities). In some embodiments, the subject has PASC.

The present disclosure is not limited to a particular level of sPLA2 (e.g., sPLA2-IIA). In some embodiments, the elevated level of sPLA2 (e.g., sPLA2-IIA) is an elevated level relative to a reference level selected from the group consisting of the level in a subject not diagnosed with a respiratory disorder, the level of the subject prior to being diagnosed with the respiratory disorder, and a population average of subjects not diagnosed with respiratory disorders. In some embodiments, the elevated level of sPLA2 (e.g., sPLA2-IIA) is above 10, 20, 30, 40, 50, 75, 100, 150, 200, 250, 300, 350, or 400 ng/ml.

Further embodiments provide a method of treating COVID-19 or PASC in a subject, comprising: a) assaying a sample from the subject for the level of secreted phospholipase A2 isoform IIA (sPLA₂-IIA); and b) administering an sPLA2-IIA inhibitor to the subject when the sample has an elevated level of sPLA2-IIA.

Other embodiments provide a method of treating COVID-19 in a subject, comprising: a) assaying a sample from the subject for the level of sPLA2A (e.g., sPLA2-IIA) and blood urea nitrogen (BUN); and b) administering an sPLA2 inhibitor to the subject when the sample has a level of sPLA2 above 10 ng/ml and a level of BUN above 16 ng/dl.

Also provided is a method of treating post-COVID-19 syndrome in a subject, comprising: a) assaying a sample from the subject for the level of sPLA2 (e.g., sPLA2-IIA); and b) administering an sPLA2 inhibitor to the subject when the sample has a level of sPLA2 above a threshold level. In some embodiments, the assaying is repeated at one or more time points (e.g., weekly, monthly, or yearly). In some embodiments, the administering is continued until the level of sPLA2-IIA drops below the threshold level and/or the subject has a decrease in symptoms of post-COVID-19 syndrome.

Additional embodiments provide a method of providing a prognosis to a subject diagnosed with a respiratory disorder or multiple organ failure, comprising: a) assaying a sample from the subject for the level of sPLA2-IIA; and b) identifying the subject as having an increased risk of severe disease and/or death when the level of sPLA2-IIA is elevated.

Certain embodiments provide a method of providing a prognosis to a subject diagnosed with a respiratory disorder or multiple organ failure, comprising: a) assaying a sample from the subject for the level of acetylcarnitine; and b) identifying the subject as having an increased risk of severe disease and/or death when the level of acetylcarnitine is elevated.

Also provided is a method of treating a condition, disease or disorder associated with mitochondrial dysfunction in a subject, comprising: a) assaying a sample from the subject for the level of acetylcarnitine and/or mitochondrial DNA; and b) administering an sPLA2A (e.g., sPLA2-IIA) inhibitor to said subject when the sample has an elevated level of acetylcarnitine and/or mitochondrial DNA.

Yet other embodiments provide the use of a sPLA2A (e.g., sPLA2-IIA) inhibitor to treat a condition, disease, or disorder in a subject with an elevated level of one or more of sPLA2A (e.g., sPLA2-IIA), BUN, acetylcarnitine or mitochondrial DNA or an sPLA2A (e.g., sPLA2-IIA) inhibitor for use in treating a condition, disease or disorder in a subject with an elevated level of one or more sPLA2A (e.g., sPLA2-IIA), BUN, acetylcarnitine or mitochondrial DNA.

Further embodiments provide the use of a sPLA2A (e.g., sPLA2-IIA) inhibitor to treat post-COVID-19 syndrome in a subject with an elevated level of sPLA2A (e.g., sPLA2-IIA).

Additional embodiments include measuring an increased or decreased level of one or more additional markers shown in FIG. 10B.

Additional embodiments are described herein.

### BRIEF DESCRIPTION OF THE DRAWINGS

FIG. 1. Untargeted Lipidomic Analysis and COVID-19 Status. A) Volcano plots show significant alterations in the lipidome of the deceased COVID-19 patients compared with non-COVID19, mild and severe COVID-19 patients. B) Heatmap of the top 20 metabolites whose abundances varied significantly across non-COVID-19, mild, severe, and deceased COVID-19 patients. C) Abundances of two lyso-PL, two free fatty acids (FFA) and two short chain acyl carnitines extracted from the untargeted lipid data were calculated and analyzed using a one-sided Wilcoxon test. Significance indicated as: * p<0.05; ** p<0.01; *** p<0.001; **** p<0.0001. D) Model of PLA₂ reaction showing how PLA₂ hydrolyzes the sn-2 position of the glycerol backbone of phospholipids to form lyso-PL and FFA products.

FIG. 2. Association of sPLA2-IIA and COVID-19 Status. A) sPLA2-IIA levels were determined in 127 plasma samples. The inset boxplot demonstrates the medians, bottom and top quantiles, and statistical significance across all four groups. B) sPLA₂ enzymatic activity was assayed within the plasma of selected samples. C) The scatter plot depicts a linear relationship between plasma sPLA₂-IIA levels and sPLA₂ activity. D) The heatmap shows the significant associations between sPLA₂-IIA levels and clinical indices of disease severity (FDR<0.05 in Spearman correlation.

FIG. 3. A Clinical Decision Tree Predicting COVID-19 Severity and Mortality. A) The tree model. Patients are classified based on the indicated clinical indices (shown in orange diamonds) and boundary conditions (above split arrows). The number of patients following each split is shown in parentheses beneath the split arrow (patients with missing index values were not split). In each node, the percentages of patients in corresponding categories are shown. (Inset) The area under the ROC curve, AUC, of the tree in determining each group membership (e.g., deceased vs. non-deceased). B) Decision surface based on the sPLA₂ and BUN boundary conditions in A. Left (L) and right (R) graphs show the results of applying the sPLA2 and BUN boundary conditions to the L and R subsets of patients (split following the 7-category ordinal scale), as indicated in A. C) The PLA-BUN index. (Top) The accuracies of combining both decision boundary conditions of sPLA2 and BUN as in B (the PLA-BUN index) in classifying severe and deceased COVID-19 patients were compared to such classification accuracies using the single decision boundary (as in B) of either sPLA2 or BUN. (Bottom) The accuracies of the PLA-BUN index in classifying severe (x-axis) and deceased (y-axis) COVID-19 patients are indicated with a red star, which are higher than such classification accuracies of using the single index of sPLA2 (light blue curve) or BUN (dark blue curve) with varying cutoff values in the corresponding data range (sPLA2, 3.4-1101.2 ng/mL; BUN, 5-242 mg/dL).

FIG. 4. Feature importance ranking of clinical indices. The relative importance of the 80 clinical indices in separating the deceased from severe COVID-19 patients (n=30 each) was evaluated in a random forest analysis (tree number=1,000 each in 10 repeats). The importance of a feature (clinical index) was assessed by the decrease of prediction accuracy when such a feature was excluded in the model, based on the Gini impurity following a node split (A; MDI, Mean Decrease Impurity) and the permuted values of the feature (B; MDA, Mean Decrease Accuracy). The top 30 features in each importance measurement are shown (color scheme is proportional to the importance score).

FIG. 5. Roles of sPLA2-Group II in mediating COVID-19 severity and outcomes. A) sPLA₂-IIA hydrolyzes phospholipids in activated and dying cells within tissues and organs. B) sPLA₂-IIA hydrolyzes mitochondrial membranes. C) Damaged mitochondria are internalized by bystander leukocytes. D) sPLA₂-IIA hydrolyzes extracellular vesicles (EV) containing eicosanoid-producing enzymes (cyclooxygenase (COX-1), thromboxane synthase (Tx synthase) and 12-lipoygenase).

FIG. 6. Clinical indices and disease status. Clinical indices that vary significantly (FDR<0.05, F-test) across 4 patient groups are shown in a heatmap, with blue to red representing low to high values of each index, and the color intensity representing the magnitude of value (mean-centered, scaled by the standard deviation, and log-transformed with non-Gaussian distribution). Missing values are shown in grey.

FIG. 7. Targeted lysophospholipid (lyso-PL) analysis. Lyso-PLs were identified as top molecules of interest from the qualitative untargeted lipidome data set. Samples were re-analyzed utilizing lyso-PL standards in a targeted lyso-PL method. Asterisks indicate significance by ANOVA (1-way ANOVA with Dunnen multiple comparison): * p<0.05; ** p<0.01; *** p<0.001; **** p<0.0001

FIG. 8. Markers of mitochondrial dysfunction. A) ROC curves demonstrate the performance of acetylcarnitine as a predictor of disease status. B) Mitochondrial DNA was quantified in a selected subset of patients (n=34; 9 non-COVID-19, 8 mild, 7 severe, and 10 deceased COVID-19 patients). Summed copy numbers of genes for human cytochrome C and cytochrome C oxidase subunit III are shown. Data are log transformed with one-sided Wilcoxon test significance indicated as: * p<0.05; ** p<0.01; *** p<0.001.

FIG. 9. Model for lung and tissue damage in patients with COVID-19.

FIG. 10. Proteomic model for predicting patient mortality. A) Machine learning model. B) Of 5,124 circulating proteins, 21 proteins were selected including sPLA2-Group II to be associated with mortality.

FIG. 11. Kinetics of the release of sPLA2 isoforms.

### DEFINITIONS

To facilitate an understanding of the present invention, a number of terms and phrases are defined below:

As used herein, the terms “detect”, “detecting” or “detection” may describe either the general act of discovering or discerning or the specific observation of a metabolite.

As used herein, the term “subject” refers to any organisms that are screened using the methods described herein. Such organisms preferably include, but are not limited to, mammals (e.g., humans).

The term “diagnosed,” as used herein, refers to the recognition of a disease by its signs and symptoms, or genetic analysis, pathological analysis, histological analysis, and the like.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, tumors, (e.g., biopsy samples), cells, and gases. Biological samples include blood products, such as plasma, serum and the like. Such examples are not however to be construed as limiting the sample types applicable to the present invention.

A “reference level” of an analyte means a level of the analyte (e.g., sPLA2) that is indicative of a particular disease state, phenotype, or lack thereof, as well as combinations of disease states, phenotypes, or lack thereof. A “positive” reference level of an analyte means a level that is indicative of a particular disease state or phenotype. A “negative” reference level of an analyte means a level that is indicative of a lack of a particular disease state or phenotype.

A “reference level” of a metabolite may be an absolute or relative amount or concentration of the analyte, a presence or absence of the analyte, a range of amount or concentration of the analyte, a minimum and/or maximum amount or concentration of the analyte, a mean amount or concentration of the analyte, and/or a median amount or concentration of the analyte. Appropriate positive and negative reference levels of an analyte for a particular disease state, phenotype, or lack thereof may be determined by measuring levels of the analyte in one or more appropriate subjects, and such reference levels may be tailored to specific populations of subjects (e.g., a reference level may be age-matched so that comparisons may be made between metabolite levels in samples from subjects of a certain age and reference levels for a particular disease state, phenotype, or lack thereof in a certain age group). Such reference levels may also be tailored to specific techniques that are used to measure levels of the analyte in biological samples (e.g., LC-MS, GC-MS, etc.), where the levels of the analyte may differ based on the specific technique that is used.

As used herein, the term “cell” refers to any eukaryotic or prokaryotic cell (e.g., bacterial cells such as E. coli, yeast cells, mammalian cells, avian cells, amphibian cells, plant cells, fish cells, and insect cells), whether located in vitro or in vivo.

“Mass Spectrometry” (MS) is a technique for measuring and analyzing molecules that involves fragmenting a target molecule, then analyzing the fragments, based on their mass/charge ratios, to produce a mass spectrum that serves as a “molecular fingerprint”. Determining the mass/charge ratio of an object is done through means of determining the wavelengths at which electromagnetic energy is absorbed by that object. There are several commonly used methods to determine the mass to charge ration of an ion, some measuring the interaction of the ion trajectory with electromagnetic waves, others measuring the time an ion takes to travel a given distance, or a combination of both. The data from these fragment mass measurements can be searched against databases to obtain definitive identifications of target molecules. Mass spectrometry is also widely used in other areas of chemistry, like petrochemistry or pharmaceutical quality control, among many others.

### DETAILED DESCRIPTION OF THE INVENTION

Both pathogen burden and health of the host are paramount to mounting a successful defense against infection by a pathogen such as SARS-CoV-2. Up to 80% of SARS-CoV-2-affected subjects are asymptomatic or develop mild to moderate symptoms; however, others progress to severe and life-threatening disease, requiring hospitalization and specialized medical care.

Early studies suggested that the host response to SARS-CoV-2 is driven by a dysregulated immune response known as cytokine storm syndrome (CSS) that underlies much of the etiology of respiratory failure and related complications. However, more recent studies reveal that the CSS is a relatively rare (3-4%) in severe COVID-19 patients, and high dose steroids such as dexamethasone only benefit a small proportion of Individuals with severe disease. In fact, there is now evidence for that immunosuppression and not the CSS compromises host immunity leading to unrestrained viral replication and severe COVID-19. Other studies highlight the major gaps remain in understanding of the underlying biology responsible for the tissue/organ damage and other immune pathologies observed in severe/lethal COVID-19, even after the viral burden has been decreased.

The role of lipid metabolism in regulating host resistance or disease tolerance after the SARS-CoV-2 infection remains unknown. Several lipidomic studies indicate that severe COVID-19 infection modifies the circulating lipidome, suggesting that dysregulated lipid metabolism contributes to disease severity. Specifically, increased severity is associated with markedly decreased plasma and exosomal levels of several unsaturated fatty acid (UFA)-containing-phospholipids, and increases in lyso-phospholipids, unesterified UFAs, and UFA containing triacylglycerides. Collectively, this biochemical pattern support that a cellular or circulating phospholipase A2 (sPLA2) that cleaves intact phospholipids from membranes forming lyso-phospholipids and unesterified UFAs may be central to COVID-19 disease.

The secreted phospholipase A2 (sPLA2) family includes 12 members (e.g., PLA2G12B, PLA2G1B, PLA2G16, PLA2G5, PLA2G10, PLA2G2C, PLA2G2E, PLA2G7 and PLA2G2D) and has several conserved characteristics, which include their low molecular weight (14-16 kDa), their requirement for high Ca2⁺ levels for catalytic activity, and the presence of a histidine and aspartic acid dyad in their catalytic site. Group IIA sPLA2 (sPLA2-IIA) known as “non-pancreatic”, “synovial”, “platelet”, “inflammatory”, and “bactericidal” is the most studied sPLA2. Initially identified in circulation and synovial fluid of rheumatoid arthritis patients, sPLA2-IIA elevations occur in a variety of clinical conditions, including sepsis and systemic bacterial infections, adult respiratory disease syndrome (ARDS), atherosclerosis, cancer, and multiple organ trauma. During a human infection, sPLA2-IIA and other secreted PLA2 isoforms can be released from numerous activated cells including endothelial cells, platelets, haptic and smooth muscle cells, and a wide range of inflammatory cells. Human bacterial infection can induce levels of sPLA2-IIA that reach plasma concentrations of 250-500ng/m1 in the acute phase, and these concentrations are capable of killing gram-positive bacteria enhancing disease resistance.

High concentrations of sPLA2 isoforms can also affect host tolerance by numerous mechanisms that lead to noxious pro-inflammatory and pro-thrombotic responses and organ dysfunction. These mechanisms include the capacity of sPLA2 to bind to cellular surfaces through receptor and non-receptor mechanisms, to hydrolyze membrane phospholipids during apoptosis or necrosis of inflammatory cells, and to hydrolyze extracellular vesicles with enzymatic machinery to produce pro-inflammatory eicosanoids. Activated Inflammatory cells and damaged organs and tissues can extrude intact mitochondria from the cell. Given the Alphaproteobacterial origin of mitochondria, sPLA2-IIA retains the properties needed to potently hydrolyze mitochondrial membranes and release highly pro-inflammatory damage associated molecular pattern (DAMPs) that include mitochondrial DNA, N-formyl protein, cytochrome C and cardiolipins. Increased levels of mitochondrial DNA correlate with tissue damage, disease progression, and the onset of multi-organ failure in patients affected by sepsis and ARDS and, more recently, COVID-19 disease severity. Without being limited to a particular mechanism, it is further contemplated that increased levels of mitochondrial DNA and elevated levels of sPLA2-IIA (including other secreted PLA2 isoforms) are associated with nerve and muscle damage associated with PACS. In particular, the capacity of elevated secreted PLA2 isoforms to potential destroy neuromuscular junctions and pulmonary surfactant may play a key pathobiological role in the fatigue/malaise and dyspnea seen with PASC.

Experiments conducted during the course of development of embodiments of the present disclosure identified similar lipidomic patterns of PLA2 products as those recently observed. Strikingly, very high levels of circulating sPLA2-IIA are found in moderate and severe cases and most particularly in patients that succumbed to the COVID-19 disease. Importantly, this sPLA2-IIA remains catalytically active, is strongly associated with circulating mitochondrial DNA, is highly correlated with several COVID-19 clinical parameters and fits into a clinical tree for assessing the risk of sPLA2-IIA in different COVID-19 patient groups.

Accordingly, provided herein are compositions and methods for blocking sPLA2 (e.g., sPLA2-IIA) (e.g., to treat COVID-19 and other disorders associated with elevated sPLA2-IIA. The present disclosure is not limited to a particular sPLA2. In some embodiments, the sPLA2 is a low MW, Ca⁺⁺ dependent secreted phospholipase A2 (such as sPLA2-IIA or other secreted isoforms (e.g., PLA2G12B, PLA2G1B, PLA2G16, PLA2G5, PLA2G10, PLA2G2C, PLA2G2E, PLA2G7 or PLA2G2D)).

For example, in some embodiments, provided herein is a method of treating a condition, disease or disorder in a subject, comprising: a) assaying a sample from said subject for the level of secreted phospholipase A2 (sPLA2-IIA); and b) administering an sPLA2-IIA inhibitor to the subject when the sample has an elevated level of sPLA2-IIA. In some embodiments, the patient also has a blood urea nitrogen (BUN) level greater than or equal to 16 mg/dl. In some embodiments, a combination of sPLA2-IIA and BUN levels are used to provide a prognosis and/or treat the condition, disease, or disorder.

In some embodiments, the sample is blood or a blood product (e.g., plasma). In some embodiments, the method further comprises assaying one or more of the subject's respiration rate, oxygen saturation or pulmonary lesion progression.

The present disclosure is not limited to a particular method of assaying the level of sPLA2-IIA. Examples include, but are not limited to, immunoassays (e.g., ELISA assays, fluorescent immunoassay, chemiluminescent immunoassay, radioimmunoassay, colorimetric enzyme activity assay, and fluorometric enzyme activity assay.

Any suitable sPLA2 (e.g., sPLA2-IIA) inhibitor may be utilized in the methods described herein. Examples include but are not limited to, antibodies, nucleic acids (e.g., antisense nucleic acids, siRNAs, shRNAs, miRNAs, etc.), and small molecules (e.g., varespladib methyl, AZD2716, 7,7-Dimethyleicosadienoic Acid (DEDA), oleyloxyethyl phosphorylcholine, luffariellolide, thioetheramide PC, 4-[(1-oxo-7-phenylheptyl)amino]-(4R)-octanoic acid, LY315920, or γS-[(1-oxo-7-phenylheptyl)amino]-4-(phenylmethoxy)-benzenepentanoic acid).

The present disclosure is not limited to particular conditions, diseases, or disorders. Examples include but are not limited to, a respiratory disorder, a trauma, a bacterial infection, septic shock, heart failure, or disseminated intravascular coagulation. In some embodiments, the respiratory disorder is or includes acute respiratory distress syndrome (ARDS). In some embodiments, the subject is infected with or has been infected with the SARS-CoV-2 virus. In some embodiments, the subject has one or more symptoms of COVID-19.

The compositions and methods described herein find use in treating both acute COVID-19 and post-COVID-19 syndrome (e.g., persistent fatigue and/or muscle and nerve dysfunction following an active COVID-19 infection). In some embodiments, the assaying is repeated at one or more time points (e.g., weekly, monthly, or yearly). In some embodiments, the administering is continued until the level of sPLA2-IIA drops below the threshold level and/or the subject has a decrease in symptoms of post-COVID-19 syndrome or COVID-19.

In some embodiments, the subject is at increased risk of severe disease or death from the disorder (e.g., due to age over 65 or comorbidities).

The level of sPLA2-IIA is compared to reference levels using various techniques, including a simple comparison (e.g., a manual comparison) of the level(s) of sPLA2-IIA in the biological sample to reference levels (e.g., the level in a subject not diagnosed with a respiratory disorder). The level may also be compared to reference levels using one or more statistical analyses (e.g., t-test, Welch's T-test, Wilcoxon's rank sum test, random forest).

The present disclosure is not limited to a particular reference level of sPLA2-IIA. In some embodiments, the elevated level of sPLA2-IIA is an elevated level relative to a reference level selected from the group consisting of the level in a subject not diagnosed with a respiratory disorder, the level of the subject prior to being diagnosed with the respiratory disorder, and a population average of subjects not diagnosed with respiratory disorders. In some embodiments, the elevated level of sPLA2-IIA is above 10, 20, 30, 40, 50, 100, 150, 200, 250, 300, 350, or 400 ng/ml. In some embodiments, the patient also has a blood urea nitrogen (BUN) level greater than or equal to 16 mg/dl.

Additional embodiments provide a method of providing a prognosis to a subject diagnosed with a respiratory disorder or disorder associated with multiple organ failure, comprising: a) assaying a sample from the subject for the level of sPLA2-IIA or acetylcarnitine; and b) identifying the subject as having an increased risk of severe disease and/or death when the level of sPLA2-IIA or acetylcarnitine is elevated.

Any patient sample suspected of containing sPLA2-IIA is tested according to the methods described herein. By way of non-limiting examples, the sample may be blood, urine, or a fraction thereof (e.g., plasma, serum, urine supernatant, urine cell pellet, urine sediment, or prostate cells).

In some embodiments, the patient sample undergoes preliminary processing designed to isolate or enrich the sample for sPLA2-IIA. A variety of techniques may be used for this purpose, including but not limited: centrifugation; immunocapture; and cell lysis.

sPLA2-IIA may be detected using any suitable method including, but not limited to, liquid and gas phase chromatography, alone or coupled to mass spectrometry (See e.g., experimental section below), NMR (See e.g., US patent publication 20070055456, herein incorporated by reference), immunoassays, chemical assays, spectroscopy and the like. In some embodiments, commercial systems for chromatography and NMR analysis are utilized.

In other embodiments, sPLA2-IIA is detected using optical imaging techniques such as magnetic resonance spectroscopy (MRS), magnetic resonance imaging (MRI), CAT scans, ultra sound, MS-based tissue imaging or X-ray detection methods (e.g., energy dispersive x-ray fluorescence detection).

Any suitable method may be used to analyze the biological sample in order to determine the presence, absence or level(s) of sPLA2-IIA. Suitable methods include chromatography (e.g., HPLC, gas chromatography, liquid chromatography), mass spectrometry (e.g., MS, MS-MS), enzyme-linked immunosorbent assay (ELISA), antibody linkage, other immunochemical techniques, biochemical or enzymatic reactions or assays, and combinations thereof. Further, the level(s) of the one or more metabolites may be measured indirectly, for example, by using an assay that measures the level of a compound (or compounds) that correlates with the level of the biomarker(s) that are desired to be measured.

In some embodiments, a computer-based analysis program is used to translate the raw data generated by the detection assay (e.g., the presence, absence, or amount of a sPLA2) into data of predictive value for a clinician. The clinician can access the predictive data using any suitable means. Thus, in some embodiments, the present invention provides the further benefit that the clinician, who is not likely to be trained in metabolite analysis, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information provides, medical personal, and subjects. For example, in some embodiments of the present invention, a sample (e.g., a blood, urine or plasma sample) is obtained from a subject and submitted to a profiling service (e.g., clinical lab at a medical facility, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (e.g., sPLA2-IIA level), specific for the diagnostic or prognostic information desired for the subject.

The profile data is then prepared in a format suitable for interpretation by a treating clinician. For example, rather than providing raw data, the prepared format may represent a diagnosis or risk assessment (e.g., prediction of the severity of respiratory disease) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may choose further intervention or counseling based on the results. In some embodiments, the data is used for research use. For example, the data may be used to further optimize the inclusion or elimination of markers as useful indicators of a particular condition or stage of disease.

When the amount(s) or level(s) of sPLA2-IIA in the sample are determined, the amount(s) or level(s) may be compared to reference levels, such as the levels in healthy individuals to aid in diagnosing or to diagnose whether the subject has severe respiratory disease. Levels of the one or more metabolites in a sample corresponding to the reference levels (e.g., levels that are the same as the reference levels, substantially the same as the reference levels indicative of severe disease, above and/or below the minimum and/or maximum of the reference levels, and/or within the range of the reference levels) are indicative of a diagnosis, risk, or prognosis of severe disease in the subject. Levels of the one or more metabolites in a sample corresponding to reference levels below the level associated with severe disease (e.g., levels that are the same as the reference levels, substantially the same as the reference levels, above and/or below the minimum and/or maximum of the reference levels, and/or within the range of the reference levels) are indicative of a diagnosis of mild or moderate respiratory disease in the subject.

In some embodiments, quantitative reference levels for a specific diagnosis or prognosis are determined and utilized to provide a risk assessment, diagnosis, prognosis, or treatment.

### EXPERIMENTAL

### Example 1

### Methods

Study Design

This retrospective study analyzed 127 plasma samples from patients hospitalized at Stony Brook University Medical Center (Stony Brook, NY, United States) from January to July 2020. This study followed Good Clinical Practice guidelines and was approved by the central institutional review board at Stony Brook University (IRB2020-00423). COVID-19 was diagnosed using the viral nucleic acid test (RT-PCR) per guidelines from Centers for Disease Control and Prevention (CDC). COVID-19 patients were classified into 3 groups: 1) mild=mild symptoms without pneumonia on imaging and discharged from inpatient care, 2) severe=respiratory tract or non-specific symptoms, pneumonia confirmed by chest imaging, oxygenation index below 94% on room air, and discharged from inpatient care, 3) deceased=expired during inpatient care.

Sample Processing and Lipidomic Analyses

Frozen EDTA plasma samples were processed utilizing Biosafety Level 2 conditions as per CDC Guidelines for the handling and processing of specimens associated with Corona Virus Disease 2019. Metabolites were isolated from plasma via methanol-based containing 10 μl Splash Lipidomix (#330707, Avanti Polar Lipids, Alabaster, AL) and separated utilizing a reverse phase chromatography as previously described by Najdekr et al. (Najdekr L, Blanco G R, Dunn W B. Collection of Untargeted Metabolomic Data for Mammalian Urine Applying HILIC and Reversed Phase Ultra Performance Liquid Chromatography Methods Coupled to a Q Exactive Mass Spectrometer. Methods Mol Biol 2019; 1996:1-15). Samples were analyzed utilizing an UHPLC-ESI-MS/MS system (UHPLC, Thermo Horizon Vanquish Duo System, MS, Thermo Exploris 480) and separation was achieved utilizing an Hypersil GOLD aQ UPLC column (100×2.1 mm, 1.9 μm, Thermo Fisher Scientific, Part No. 25302-102130) with mobile phases composed of water containing 0.1% formic acid and methanol containing 0.1% formic acid. Metabolites were eluted over a 15 min gradient with the Exploris 480 operating in positive ion mode, utilizing an ion transfer tube temperature of 350° C., sheath gas of 45, aux gas of 5, and spray voltage of 4000. MS data for all samples were collected using dynamic exclusion and then aligned with pooled samples collected using Thermo AquireX to achieve optimal metabolite identification in Lipid Search 4.0 and Thermo Compound Discoverer 2.3 software. Untargeted lipidomic data were transformed, normalized, and analyzed using MetaboAnalyst 4.0. The Benjamini-Hochberg procedure was used to control the false discovery rate (FDR), and the molecules with FDR≤0.1 and absolute log2 fold change (FC)≥1.5 were considered as significant and biologically relevant. Individual metabolites were compared between groups with one-sided Mann-Whitney Wilcoxon tests at an alpha level of 0.05.

Targeted lipidomic analysis was performed using an Agilent 1200 HPLC tandem Thermo Quantum Ultra triple quadrupole mass spectrometer (Thermo Fisher Scientific, San Jose, USA) to quantify levels of major molecular species of lyso-phospholipids (lyso-PLs). C16, C18:1, C18:2, and C20:4 molecular species for lyso-phosphatidylcholine (lyso-PC), lyso-phosphatidylethanolamine (lyso-PE), and lyso-phosphatidylserine (lyso-PS) (Cayman Chemical, Ann Arbor, MI) were used as standards and deuterated Splash Lipidomix as internal standards. Lyso-PLs were separated using an Agilent Poroshell 120 EC-C18 1.9 μm (2.1×50 mm) with mobile phases composed of water containing 2 mM ammonium formate/0.1% formic acid (A) and methanol containing 1 mM ammonium formate/0.1% formic acid. Chromatographic gradient elution began at 40% A and remained there for the first minute, proceeding to 1% A at 6 minutes, staying there for 10.5 min before returning to 40% MPA over 1.5 min and remaining till the end of the 20 min run.

sPLA₂-IIA Concentrations

sPLA₂-IIA levels in plasma were determined by ELISA (Cayman Chemical Company). Plasma samples were diluted (1:20-1:800) and assayed in duplicate. Concentrations of sPLA₂-IIA in plasma were calculated using standard curves.

Enzymatic Assay for Spla₂ Activity

sPLA2 activity was assayed by modifying techniques from Kramer and Pepinsky (Kramer R M, Pepinsky R B. Assay and purification of phospholipase A2 from human synovial fluid in rheumatoid arthritis. Methods Enzymol 1991; 197:373-81). Hydrolytic activity was determined in plasma samples from 34 patients (9 non-COVID-19, 8 mild, 7 severe, and 10 deceased COVID-19 patients) representing a wide range of sPLA₂-IIA levels. Assays contained 5 μl of plasma in a final volume of 400 μl containing 50 mM Tris/NaCl, pH 8.5, with 5 mM CaCl₂ and 5 nmol of 3H-oleate-labeled E. coli phospholipids and incubated for 30 mins at 37° C.² Lipids were extracted utilizing a modified Bligh and Dyer (Bligh E G, Dyer W J. A rapid method of total lipid extraction and purification. Can J Biochem Physiol 1959; 37:911-7), and hydrolyzed fatty acids were separated from phospholipids using thin layer chromatography (Silica Gel G) and a mobile phase of hexane:ether:formic acid (90:60:6, v:v:v), and visualized by iodine vapor relative to cold standards.

Mitochondrial DNA Quantification

Mitochondrial DNA (mtDNA) was quantified by adapting methods from Scozzi et. al. (Scozzi D, Cano M, Ma L, et al. Circulating mitochondrial DNA is an early indicator of severe illness and mortality from COVID-19. JCI Insight 2021). Using genes for human cytochrome C (MT-CYB) and cytochrome C oxidase subunit III (MT-COX3), mtDNA was quantified in plasma samples from the same 34 patients (9 non-COVID-19, 8 mild, 7 severe, and 10 deceased COVID-19 patients) as in the sPLA2 activity assay utilizing an ABI 7900HT real-time PCR instrument in 384-well format. Synthetic oligonucleotide copies of the MT-CYB and MT-COX3 genomic sequences (gBlock Gene Fragments from Integrated DNA Technologies) were included to generate a standard curve at 10⁵, 10⁴, 10³, and 10² copies per μL. Primer sequences were as follows:

(SEQ ID NO: 1)
forward MT-CYB: 5′-ATGACCCCAATACGCAAAA-3′
(SEQ ID NO: 2)
reverse MT-CYB: 5′-CGAAGTTTCATCATGCGGAG-3′
(SEQ ID NO: 3)
forward MT-COX3: 5′-ATGACCCACCAATCACATGC-3′
(SEQ ID NO: 4)
reverse MT-COX3: 5′-ATCACATGGCTAGGCCGGAG-3′

Each diluted serum sample was compared to a control reaction of a gBlock standard, and the delta-Ct was used to correct the calculated concentrations from triplicate reactions.

Statistical Analyses

sPLA₂ levels, sPLA₂ activity, and mtDNA levels were compared between groups with non-parametric Mann-Whitney Wilcoxon tests at an α-level of 0.05. Spearman correlations between sPLA₂ levels and clinical indices were computed in R. Receiver operating characteristic (ROC) curves, area under the curves (AUC), and confidence intervals were generated using the R packages ROCR and pROC. A clinical decision tree was constructed using the Classification and Regression Trees (CART) algorithm (Breiman L, Friedman J, Olshen R, Stone C. Classification and Regression Trees: Chapman & Hall; 1984) implemented in the R package RPART, and the importance of individual features was ranked using random forest analysis using the R package Random Forest.

Decision Tree and Random Forest Construction

A clinical decision tree was constructed using the Classification and Regression Trees (CART for short) algorithm (Breiman L, Friedman J, Olshen R, Stone C. Classification and Regression Trees: Chapman & Hall; 1984) implemented in the R package RPART. Specifically, 80 initial clinical indices were used as input variables in decision tree learning to build a predictive model (i.e., classification tree) by recursive partitioning. The tree model identified a set of predictive features (branch conditions) that best classified the 127 patients into the 4 groups: non-COVID-19, mild, severe, and deceased COVID-19 patients. The tree split points were determined by the Gini index with the minimum leaf size=10. A tenfold cross-validation method was used to tune the tree model and evaluate its prediction accuracy. To avoid overfitting, the tree was pruned back to the smallest size while minimizing the cross-validated error. The classification accuracy of the tree to determine each group membership (e.g., deceased vs. non-deceased) was assessed using the area under the ROC curve. To evaluate the relative feature importance in accurately splitting the tree nodes between severe and deceased COVID-19 patients, a random decision forest analysis was performed using the R package randomForest (Breiman L. Random Forests. Machine Learning 2001; 45:5-32). An assembly of 5,000 random decision trees was constructed in the forest, and the importance of a given feature (i.e., one of the 80 clinical indices) was assessed by the decrease of prediction accuracy when such a feature was omitted in the model, based on the Gini impurity following a node split and an estimate of the loss in prediction performance.

### RESULTS

Patients

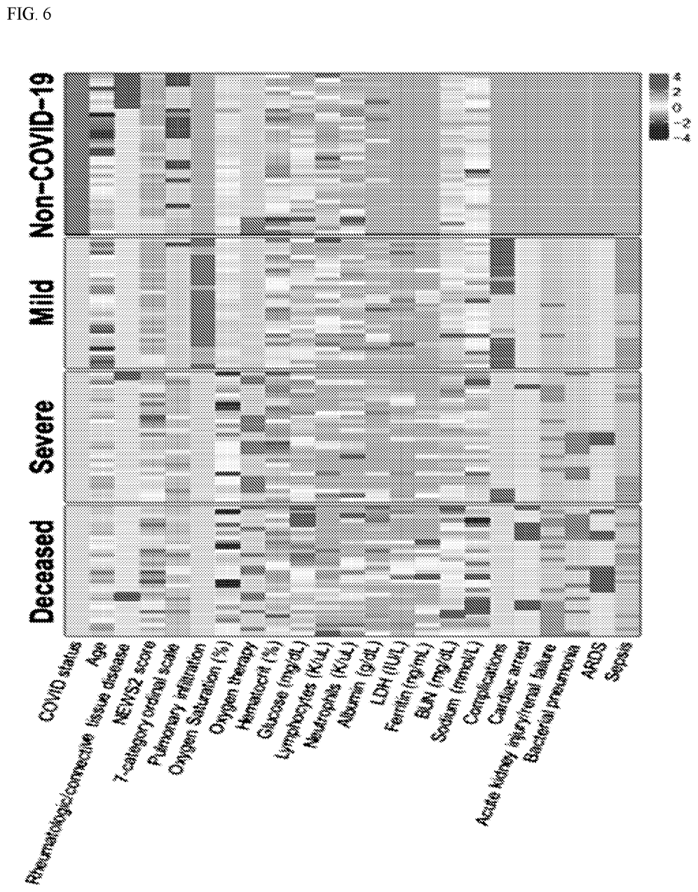

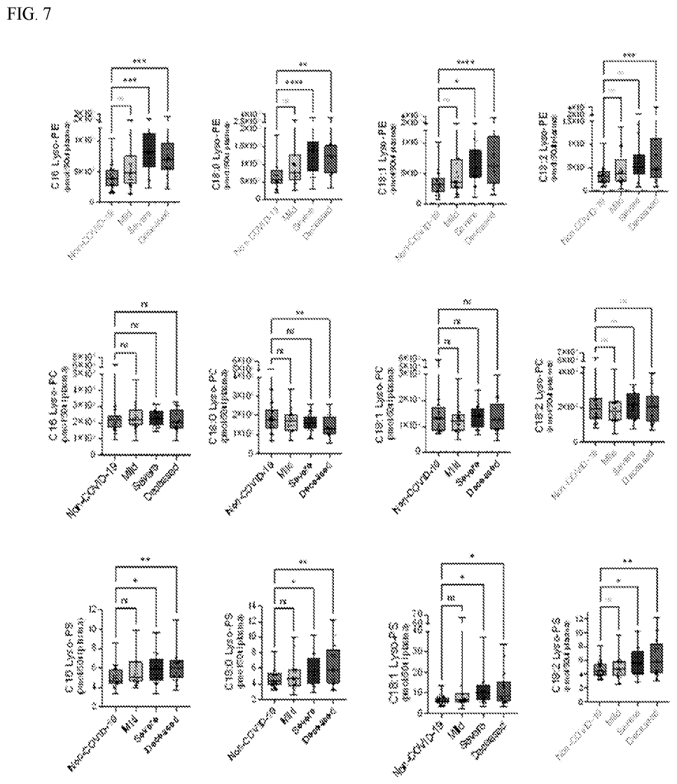

A total of 127 patient plasma samples collected between May and July 2020 were analyzed. The demographics and baseline clinical characteristics of the patients are shown in Table 1. Age differed across groups with deceased COVID-19 being older on average (FIG. 6). There were no significant trends in BMI or obesity. Prevalence of various co-morbidities was comparable across groups, except for rheumatologic disease in mild COVID-19 patients (FIG. 6). Severe and deceased COVID-19 patients experienced more complications, with higher incidences of cardiac arrest, acute kidney injury/renal failure, bacterial pneumonia, ARDS, and sepsis (FIG. 6).

Plasma Lipidomic Profiles and COVID-19 Disease Status

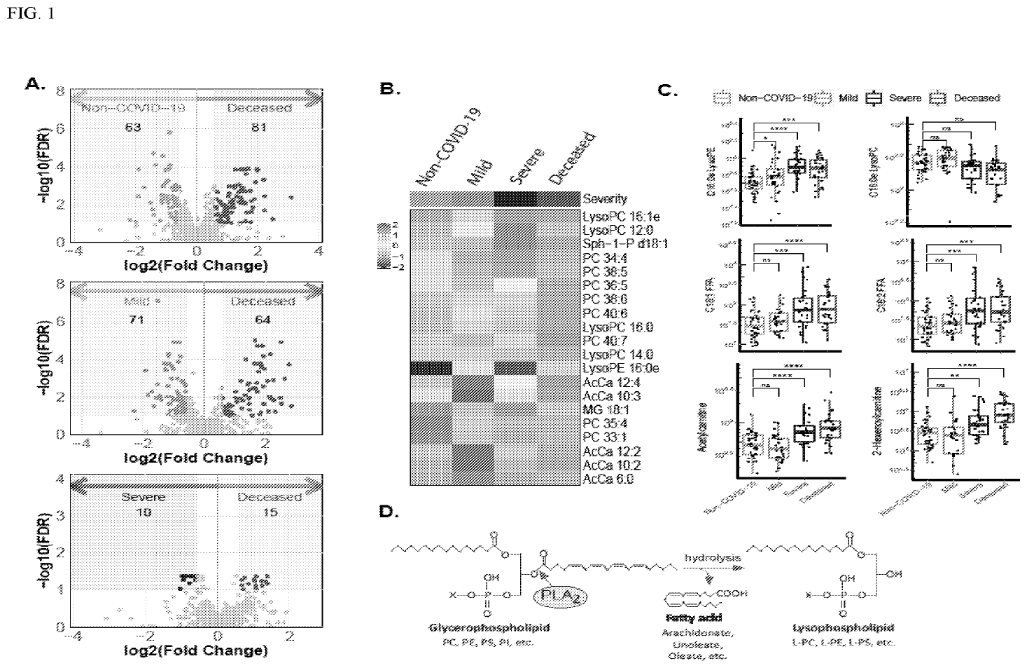

Untargeted lipidomic analysis of the plasma samples revealed that the most significant changes in the lipid profile occurred in deceased COVID-19 patients (FIG. 1A), with 181 unique molecules identified. Further analysis of the 20 most significant molecules demonstrated an enrichment in metabolites associated with acylcarnitine and phospholipid metabolism (FIG. 1B). Initial analysis showed that several lyso-phosphatidylethanolamine (lyso-PE) molecular species typified by C16eLysoPE and unsaturated fatty acids such as linoleic (18:2) and oleic acids (18:1) were elevated in severe/deceased COVID-19 patients (FIG. 1C). Targeted lipidomics confirmed the compositional untargeted lipidomic analysis, showing significant increases in major molecular species of lyso-PE and lyso-phosphatidylserine (lyso-PS) (FIG. 7) while demonstrating no changes in lyso-phosphatidylcholine (lyso-PC). Together, this supported hydrolysis by a PLA₂ activity (FIG. 1D).

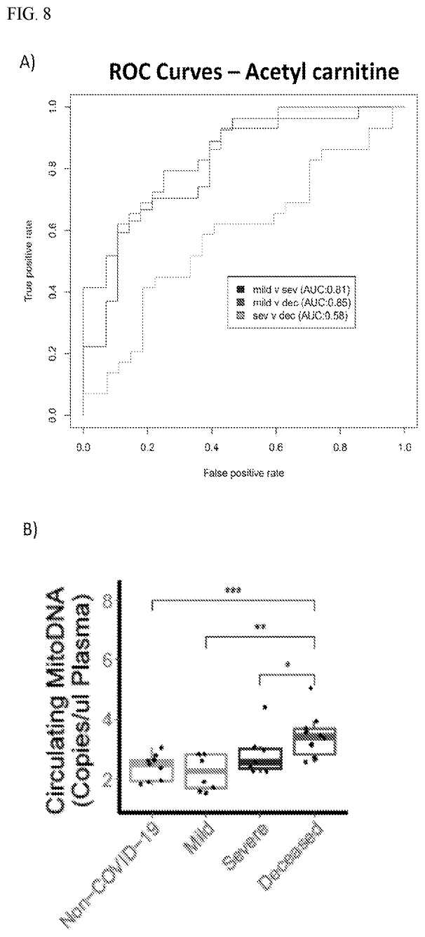

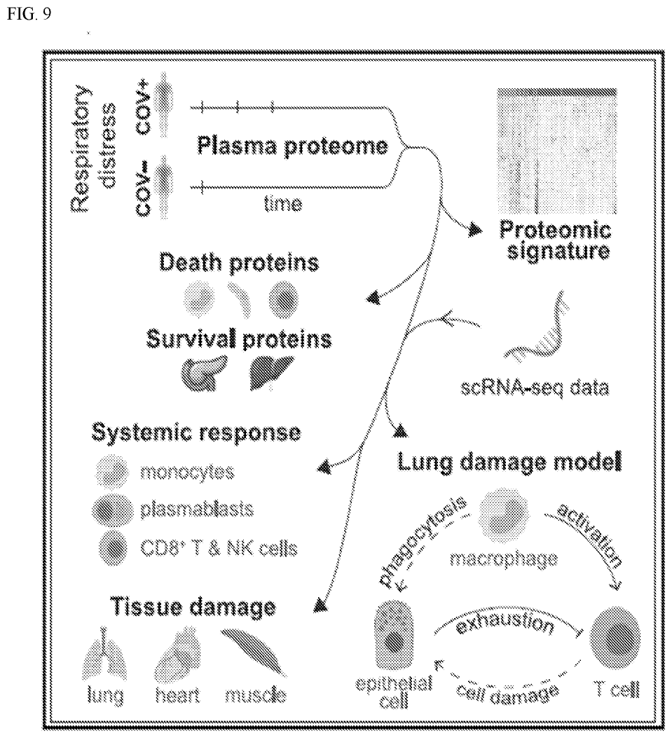

Short- and medium-chain acylcarnitines (acetyl and hexanoyl carnitines) were also elevated in severe and deceased COVID-19 patients (FIG. 1C). Plasma short chain acyl carnitine and particularly acetylcarnitine has recently been shown to serve as an independent prognostic biomarker for mortality in sepsis and heart failure (Chung K P, Chen G Y, Chuang T Y, et al. Increased Plasma Acetylcarnitine in Sepsis Is Associated With Multiple Organ Dysfunction and Mortality: A Multicenter Cohort Study. Crit Care Med 2019; 47:210-8). Furthermore, acetylcarnitine showed high areas under ROC curves: 0.810 (95% CI, 0.694-0.925) for mild vs. severe and 0.849 (95% CI, 0.752-0.945) for mild vs. deceased (FIG. 8A). Additionally, plasma concentrations of MT-CYB and MT-COX3 were significantly elevated in deceased COVID-19 patients compared to non-COVID-19 and mild COVID-19 patients, indicating elevated mtDNA (FIG. 8B).

Circulating Secreted PlA₂-IIa Associated with COVID-19 Disease Status

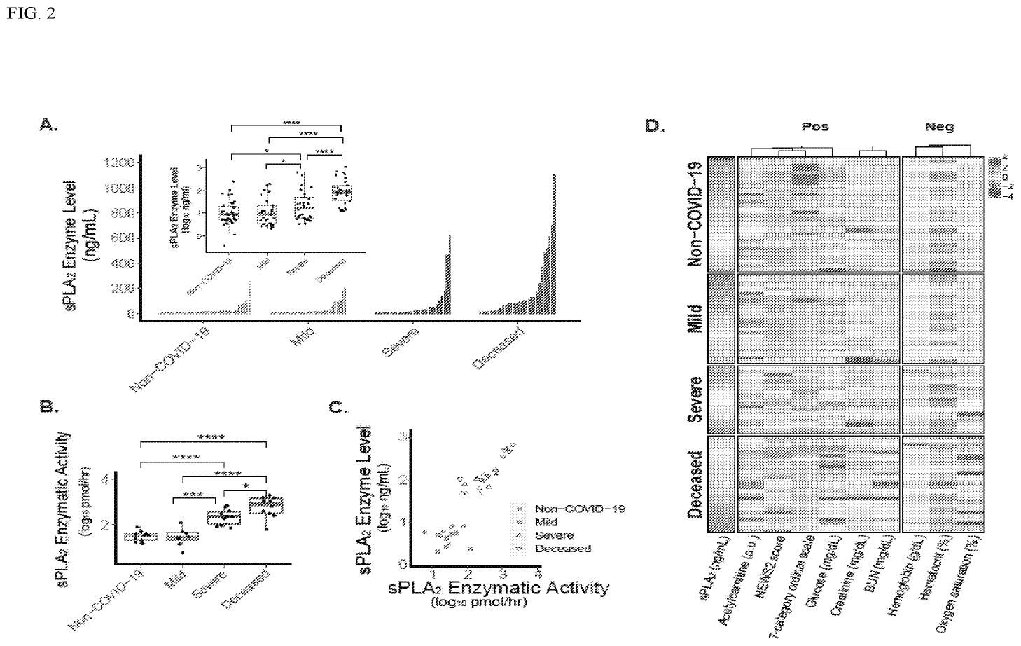

Given the critical role of sPLA₂-IIA in several related diseases (Dore et al.), its levels were quantified in all 127 patients. FIG. 2A shows the distribution of sPLA₂-IIA in all patients and the marked increase of sPLA₂-IIA in severe (66.6±25.2 ng/ml) and deceased COVID-19 patients (187.3±46.6 ng/ml) compared to non-COVID-19 (24.1±7.4 ng/ml) and mild COVID-19 patients (31.5±9.4 ng/ml). There was heterogeneity among the severe patients with 48% of severe patients having relatively normal (<10 ng/ml) levels of circulating sPLA₂-IIA levels (inset, FIG. 2A). In contrast, sPLA₂-IIA in all deceased COVID-19 patients exceeded 10 ng/ml, with 46% of these patients having at least 10-fold higher levels (ranging from 102 ng/ml to 1020 ng/ml). Enzymatic assays showed sPLA₂-IIA was catalytically active (FIG. 2B), with a strong correlation (r²=0.84, p=1.2×10⁻¹³) between sPLA₂-IIA levels and enzymatic activity (FIG. 2C).

Elevated levels of plasma sPLA₂-IIA were significantly associated with several critical clinical indices (FIG. 2D). Its positive correlation with higher baseline NEWS2 and 7-category ordinal scale scores supports the role of sPLA₂-IIA in disease severity. The positive correlation of sPLA₂-IIA with glucose levels highlights its link to inflammation. Consistently, hyperglycemia has been reported to be an important prognostic factor for COVID-19, associated with a pro-oxidative/proinflammatory state (Ceriello A. Hyperglycemia and the worse prognosis of COVID-19. Why a fast blood glucose control should be mandatory. Diabetes Res Clin Pract 2020; 163:108186). The positive correlations with creatinine and blood urea nitrogen (BUN) levels demonstrate how sPLA₂-IIA levels may also reflect kidney dysfunction. Finally, the negative correlations with hematocrit, hemoglobin levels, and baseline oxygen saturation indicate elevated sPLA₂-IIA levels may be associated with hypoxia, anemia, and multiple organ dysfunction (Hariyanto T I, Kurniawan A. Anemia is associated with severe coronavirus disease 2019 (COVID-19) infection. Transfus Apher Sci 2020; 59:102926).

Levels of Secreted PLA₂-IIA as a Central Predictor of Covid-19 Mortality

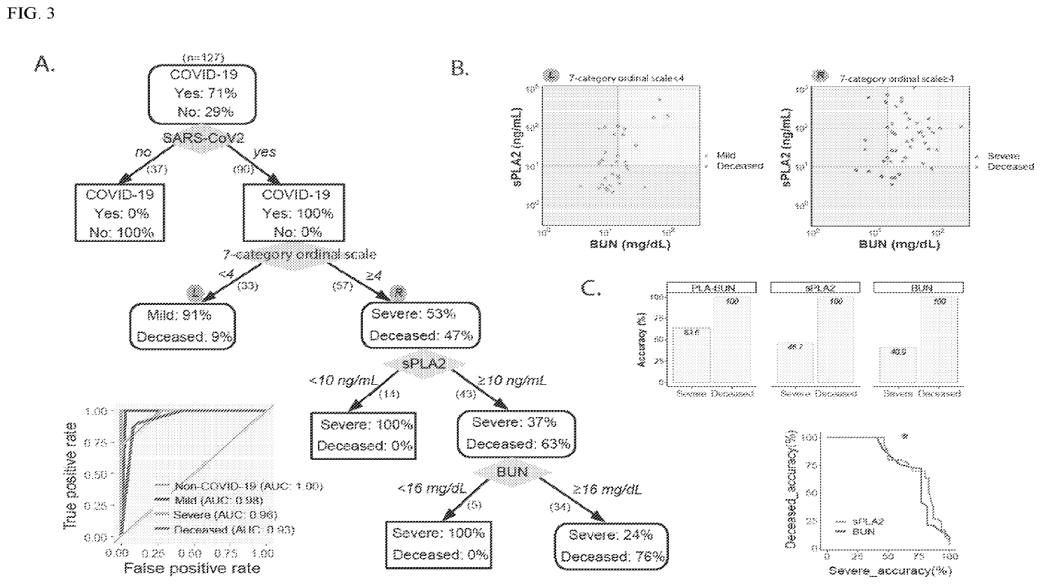

The eighty clinical indices measured in the cohort of 127 patients were analyzed by machine learning models. First, a decision tree was generated by recursive partitioning to identify critical indices that separate the four patient groups with high accuracy (area under ROC curve=0.93-1.0, FIG. 3A inset). Patients positive for COVID-19 were stratified using the predictor “7-category ordinal scale” into “mild” and “severe or deceased”, with 91% and 100% accuracy, respectively. Of patients in “severe or deceased”, sPLA₂-IIA levels were found to effectively separate the survivors from non-survivors. Those with sPLA₂-IIA<10 ng/mL were classified as “severe” but not “deceased” with a 100% accuracy; in contrast, those with sPLA₂-IIA≤10 ng/mL were placed as “deceased” with a 63% accuracy. Of these sPLA2-high (≥10 ng/mL) patients, the levels of blood urea nitrogen (BUN) further helped improve the prediction of survival: those with BUN<16 mg/dL were classified as “severe” but not “deceased” (100% accuracy); conversely, those with BUN≤16 mg/dL were markedly enriched (76%) with “deceased” patients.

In short, the decision tree identified sPLA₂ and BUN as two critical risk factors for COVID-19 mortality. Correspondingly, the effective separation of mild, severe, and deceased COVID-19 patients can be visualized in the sPLA2-BUN boundary graphs (FIG. 3B).

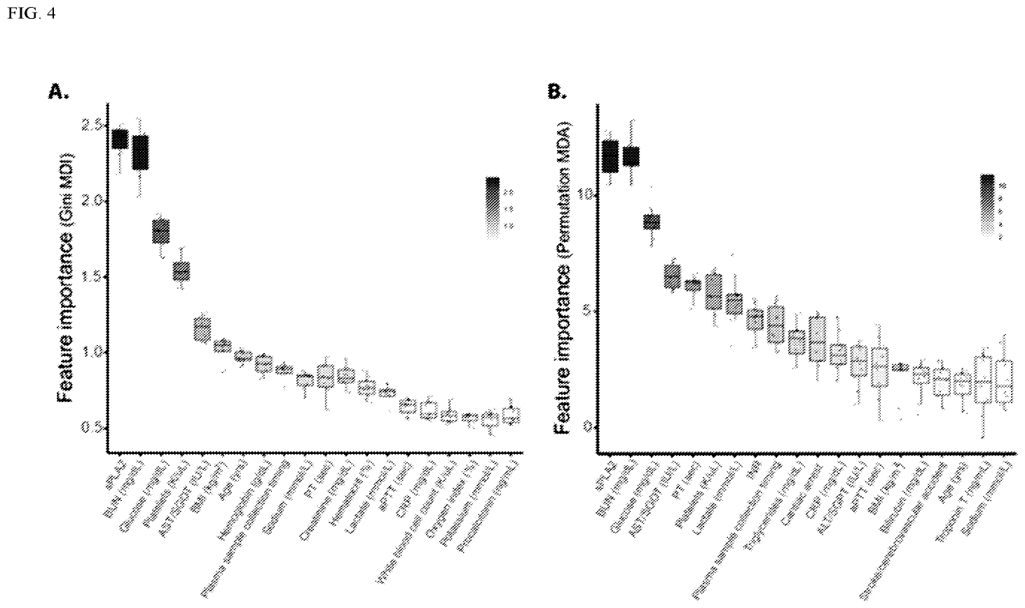

The decision tree provided the explanation and interpretation for the progression of COVID-19 disease severity, built on the patient data available. To validate the model results that placed sPLA2 and BUN as the two critical predictors of COVID-19 mortality, an additional random forest analysis was performed to evaluate the relative importance of all 80 clinical indices. In the random forest model, subsets of patients and features (clinical indices) were randomly selected to build an assembly of decision trees (1,000 trees each in 10 repeats) to provide a robust assessment of feature importance in separating severe versus deceased patients. Again, sPLA2 and BUN were found as the top 2 features ranking significantly higher (p<0.0001) than all other clinical indices to accurately predict COVID-19 mortality (based on both measures of feature importance, Gini MDI, left, and Permutation MDA, right, FIG. 4). Additionally, combining both decision boundary conditions of sPLA₂ and BUN (the PLA-BUN index) performed more accurately than using either index alone (FIG. 3C).

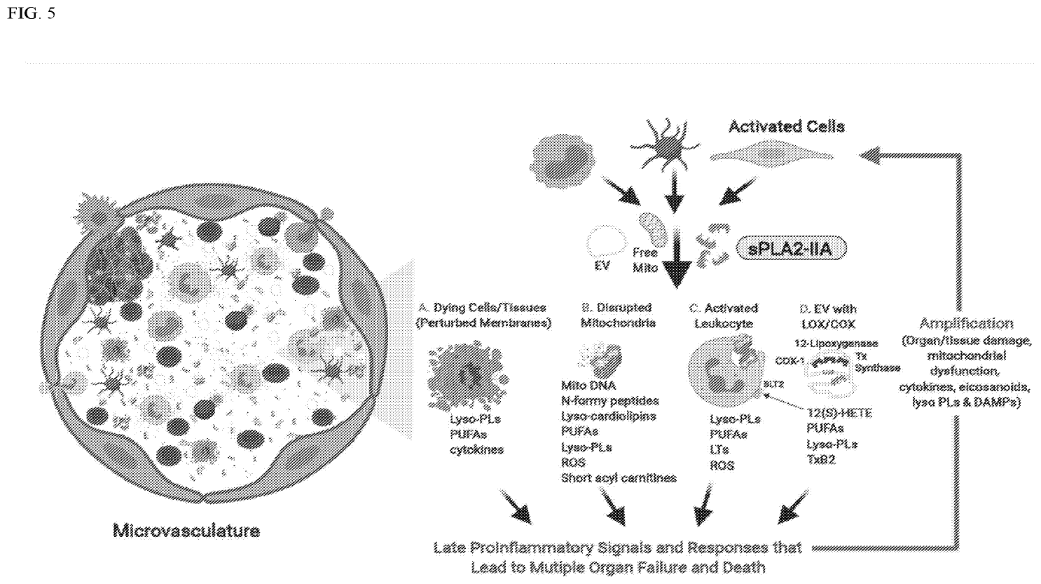

sPLA₂-IIA has direct and organism-wide pathogenic characteristics (FIG. 5) (Dore E, Boilard E. Roles of secreted phospholipase A(2) group IIA in inflammation and host defense. Biochim Biophys Acta Mol Cell Biol Lipids 2019; 1864:789-802; Boudreau L H, Duchez A C, Cloutier N, et al. Platelets release mitochondria serving as substrate for bactericidal group IIA-secreted phospholipase A2 to promote inflammation. Blood 2014; 124:2173-83; Murakami M, Sato H, Taketomi Y. Updating Phospholipase A(2) Biology. Biomolecules 2020; 10) with the capacity to impact COVID-19 severity and patient outcomes. During cell activation and initiation of multiple cell death mechanisms, anionic phospholipids PS and PE are externalized, exposing them to phospholipid hydrolysis by sPLA₂-IIA (FIG. 5A) (Atsumi G, Murakami M, Tajima M, Shimbara S, Hara N, Kudo I. The perturbed membrane of cells undergoing apoptosis is susceptible to type II secretory phospholipase A2 to liberate arachidonic acid. Biochim Biophys Acta 1997; 1349:43-54). Hydrolysis of cellular membranes can broadly invoke tissue damage and organ cell dysfunction. Additionally, activated cells and damaged tissues/organs secrete extracellular mitochondria (Boudreau et al, supra). As mitochondrial phospholipids are preferred substrates for sPLA₂-IIA, their catalysis releases mtDNA, acetylcarnitine, and several danger-associated molecular pattern (DAMPs) (FIG. 5B). Damaged mitochondria internalized by bystander leukocytes (FIG. 5C) increase inflammatory mediators including lyso-PLs, unsaturated fatty acids, eicosanoids and cytokines. sPLA₂-IIA hydrolyzes platelet-derived extracellular vesicles (EV) to release cyclooxygenase, thromboxane synthase and 12-lipoygenase inflammatory eicosanoids (Dore E, Boilard E. Roles of secreted phospholipase A(2) group IIA in inflammation and host defense. Biochim Biophys Acta Mol Cell Biol Lipids 2019; 1864:789-802). Collectively, these events amplify late inflammatory responses with the potential to further damage tissues and organs in severe and lethal COVID-19.

TABLE 1
Demographics and Clinical Characteristics at Baseline

| | Non | COVID-19 | | | |
|---|---|---|---|---|---|
| | COVID-19 | Mild | Severe | Deceased | |
| Variables | (n = 37) | (n = 30) | (n = 30) | (n = 30) | p-value |

Demographics

| | | | | | | | | | |
|---|---|---|---|---|---|---|---|---|---|
| Mean age (range) - yr | 57.08 | (10-84) | 53.37 | (14-93) | 62.4 | (35-86) | 71.17 | (48-96) | 0.0027 |

Sex: no. of patients (%)

| | | | | | | | | | |
|---|---|---|---|---|---|---|---|---|---|
| Male | 20 | (54.0) | 12 | (40.0) | 16 | (53.3) | 20 | (66.7) | 0.2314 |
| Female | 17 | (46.0) | 18 | (60.0) | 14 | (46.7) | 10 | (33.3) | |

Race/ethnicity: no. of patients (%)

| | | | | | | | | | |
|---|---|---|---|---|---|---|---|---|---|
| White | 28 | (75.7) | 19 | (63.3) | 14 | (46.7) | 19 | (63.3) | 0.0605 |
| Black or African American | 2 | (5.4) | 1 | (3.3) | 2 | (6.7) | 0 | (0.0) | |
| Asian | 1 | (2.7) | 0 | (0.0) | 0 | (0.0) | 4 | (13.3) | |
| Hispanic or Latino | 5 | (13.5) | 9 | (30.0) | 14 | (46.7) | 7 | (23.3) | |
| Other | 1 | (2.7) | 1 | (3.3) | 0 | (0.0) | 0 | (0.0) | |

Characteristics

| | | | | | | | | | |
|---|---|---|---|---|---|---|---|---|---|
| Median BMI, kg/m² (IQR) | 29.54 | (24.45-34.82) | 28.55 | (24.43-34.04) | 29.3 | (25.08-34.57) | 25.86 | (23.18-34.71) | 0.0334 |
| Median Charlson Comorbidity | 1 | (0-2.5) | 0 | (0-2.25) | 1 | (0-3) | 1 | (0-3) | 0.5738 |
| Index (IQR) | | | | | | | | | |
| Hypertension - no. of patients (%) | 18 | (48.7) | 14 | (46.7) | 21 | (70.0) | 18 | (60.0) | 0.2167 |
| Major cardiac disease* - no. of | 8 | (21.6) | 6 | (20.0) | 5 | (16.7) | 11 | (36.7) | 0.2687 |
| patients (%) | | | | | | | | | |
| Diabetes - no. of patients (%) | 7 | (18.9) | 6 | (20.0) | 9 | (30.0) | 9 | (30.0) | 0.5856 |
| Obesity^{¢} - no. of patients (%) | 16 | (43.2) | 13 | (43.3) | 12 | (40.0) | 5 | (16.7) | 0.0859 |
| Lipid disorder^{¥} - no. of patients (%) | 13 | (35.1) | 9 | (30.0) | 12 | (40.0) | 10 | (33.3) | 0.8750 |
| Kidney disease - no. of patients (%) | 5 | (13.5) | 3 | (10.0) | 7 | (23.3) | 6 | (20.0) | 0.4865 |
| Liver disease - no. of patients (%) | 2 | (5.4) | 3 | (10.0) | 1 | (3.3) | 1 | (3.3) | 0.6352 |
| Malignancy - no. of patients (%) | 7 | (18.9) | 2 | (6.7) | 2 | (6.7) | 5 | (16.7) | 0.2945 |
| Rheumatologic/connective tissue | 8 | (21.6) | 0 | (0.0) | 2 | (6.7) | 2 | (6.7) | 0.0179 |
| disease - no. of patients (%) | | | | | | | | | |
| Chronic lung disease, not asthma - | 2 | (5.4) | 2 | (6.7) | 4 | (13.3) | 6 | (20.0) | 0.2215 |
| no. of patients (%) | | | | | | | | | |
| Smoking - no. of patients (%) | 17 | (45.9) | 8 | (26.7) | 8 | (26.7) | 8 | (26.7) | 0.2161 |
| Asthma - no. of patients (%) | 3 | (8.1) | 1 | (3.3) | 4 | (13.3) | 2 | (6.7) | 0.5422 |

Presenting signs and symptoms

| | | | | | | | | | |
|---|---|---|---|---|---|---|---|---|---|
| Median NEWS2 score (IQR) | 0 | (0-1) | 0 | (0-1) | 7 | (5-9) | 7 | (5.75-9) | <0.0001 |
| Median 7-category ordinal scale (IQR) | 3 | (1-3) | 3 | (3-3) | 5 | (5-5) | 5 | (4.75-5) | <0.0001 |
| Pulmonary infiltration - no. of | 0 | (0.0) | 0 | (0.0) | 30 | (100.0) | 30 | (100.0) | <0.0001 |
| patients (%) | | | | | | | | | |
| Bilateral pulmonary infiltration- no. of | 0 | (0.0) | 0 | (0.0) | 26 | (86.7) | 27 | (90.0) | <0.0001 |
| patients (%) | | | | | | | | | |
| Oxygen saturation, % (IQR)^{§} | 98 | (97-99) | 98 | (96.75-99) | 92 | (84.25-93) | 90.5 | (81.25-95) | <0.0001 |

Oxygen modality - no. of patients (%)

| | | | | | |
|---|---|---|---|---|---|
| Room air | 33 | 30 | 17 | 21 | <0.0001 |
| Oxygen therapy | 4 | 0 | 13 | 9 | |

Symptoms - no. of patients (%)

| | | | | | | | | | |
|---|---|---|---|---|---|---|---|---|---|
| Abdominal pain | 4 | (10.8) | 5 | (16.7) | 0 | (0.0) | 2 | (6.7) | 0.1304 |
| Loss of appetite | 4 | (10.8) | 2 | (6.7) | 7 | (23.3) | 8 | (26.7) | 0.1009 |
| Chest pain | 8 | (21.6) | 5 | (16.7) | 5 | (16.7) | 4 | (13.3) | 0.8426 |
| Chills/rigors | 0 | (0.0) | 3 | (10.0) | 5 | (16.7) | 3 | (10.0) | 0.1080 |
| Confusion/delirium | 0 | (0.0) | 1 | (3.3) | 6 | (20.0) | 10 | (33.3) | 0.0002 |
| Dry cough | 0 | (0.0) | 1 | (3.3) | 5 | (16.7) | 10 | (33.3) | 0.0002 |
| Cough with sputum | 2 | (5.4) | 1 | (3.3) | 11 | (36.7) | 6 | (20.0) | 0.0008 |
| Diarrhea | 3 | (8.1) | 3 | (10.0) | 8 | (26.7) | 6 | (20.0) | 0.1399 |
| Dizziness | 5 | (13.5) | 1 | (3.3) | 0 | (0.0) | 2 | (6.7) | 0.1253 |
| Shortness of breath | 7 | (18.9) | 6 | (20.0) | 21 | (70.0) | 23 | (76.7) | <0.0001 |
| Fever | 0 | (0.0) | 1 | (3.3) | 16 | (53.3) | 15 | (50.0) | <0.0001 |
| Headache | 1 | (2.7) | 2 | (6.7) | 2 | (6.7) | 1 | (3.3) | 0.8090 |
| Malaise | 5 | (13.5) | 2 | (6.7) | 10 | (33.3) | 10 | (33.3) | 0.0157 |
| Fatigue | 3 | (8.1) | 2 | (6.7) | 10 | (33.3) | 11 | (36.7) | 0.0019 |
| Nasal congestion | 0 | (0.0) | 1 | (3.3) | 2 | (6.7) | 2 | (6.7) | 0.4356 |
| Nausea/vomiting | 4 | (10.8) | 3 | (10.0) | 4 | (13.3) | 3 | (10.0) | 0.9728 |
| Other | 22 | (59.5) | 21 | (70.0) | 8 | (26.7) | 12 | (40.0) | 0.0031 |

*Major cardiac disease: coronary artery disease, congestive heart failure, history of myocardial infarction
^{¢}Obesity defined as BMI ≥ 30 kg/m²
^{¥}Lipid disorder: hyperlipidemia, dyslipidemia, antiphospholipid syndrome
^{§}Some oxygenation indices measured while on oxygen therapy (no baseline measurement on room air).

Table 1: Demographics and Clinical Characteristics at Baseline. All categorical variables are represented as proportions (%) whereas continuous variables are reported as median (interquartile range). D'Agostino-Pearson normality test was used to assess continuous variables and determined all that had non-Gaussian distributions; Kruskal-Wallis test were then used to assess for equality of group variance. Categorical variables were compared using the chi-square test. P-values reflect comparisons of group variance; significant trends.

### Example 2

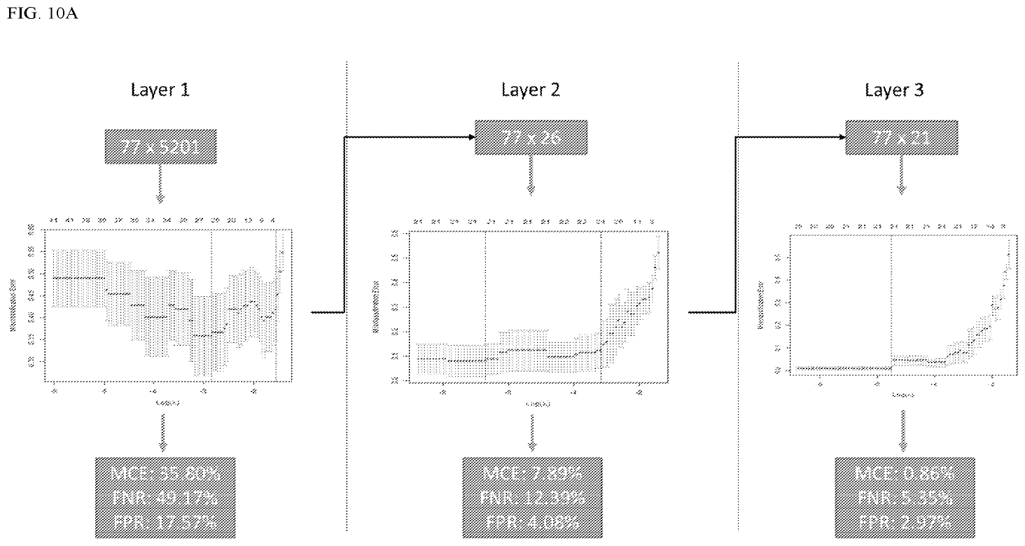

Plasma proteomics was used in 306 COVID-19 patients and 78 symptomatic controls over time to survey the role of circulating immune cells and tissue cells in inflammation, disease severity, and survival. As shown in FIG. 9, it is contemplated that interactions among myeloid, epithelial, and T cells drive tissue damage. Patient demographics are shown below:

| | | | | | Ventilated, | | Hospitalized | | Hospitalized | | Discharged | | | |
|---|---|---|---|---|---|---|---|---|---|---|---|---|---|---|
| | All COVID | | Death | | survived | | on oxygen | | no oxygen | | from ED | | Non-COVID | |
| | subjects | | (A1) | | (A2) | | (A3) | | (A4) | | (A5) | | controls | |
| Total N = 384 | 306 | | 42 | | 67 | | 133 | | 41 | | 23 | | 78 | |
| Demographics: | | | | | | | | | | | | | | |
| Age, median years [IQR] | 58 | [45-75] | 80 | [75-87] | 62 | [49-69] | 57 | [46-73] | 52 | [39-66] | 36 | [31-47] | 67 | [57-76] |
| Male, n(%) | 162 | (52.9%) | 24 | (57.1%) | 40 | (59.7%) | 70 | (52.6%) | 20 | (48.8%) | 8 | (34.8%) | 38 | (48.7%) |
| Hispanic, n(%) | 166 | (54.2%) | 12 | (28.6%) | 44 | (65.7%) | 73 | (54.9%) | 27 | (65.9%) | 10 | (43.5%) | 12 | (15.4%) |
| Black, n(%) | 30 | (9.8%) | 4 | (9.5%) | 9 | (13.4%) | 11 | (8.3%) | 6 | (14.6%) | 0 | (0%) | 3 | (3.8%) |
| BMI, median [IQR] | 29 | [26-34] | 28 | [26-35] | 29 | [26-33] | 30 | [26-35] | 27 | [25-31] | 30 | [28-32] | 26 | [23-32] |

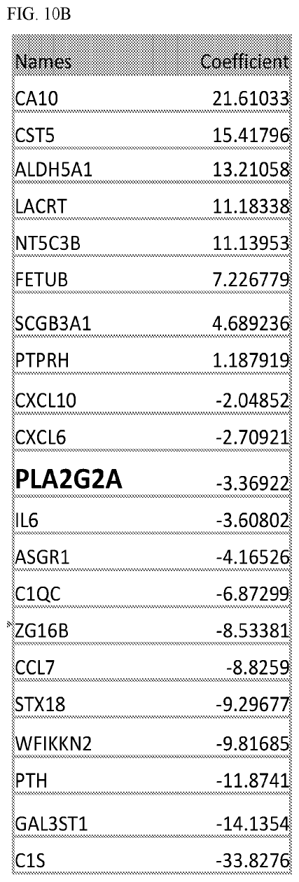

A sequential machine-learning (3-layer LASSO) approach was developed to optimize feature selection and minimize model errors from proteomic data (SomaLogic; 5,124 proteins) (FIG. 10A). This model outperformed decision tree and random forest results. The results were a 21-protein feature model for mortality (versus ICU ventilated) including PLA2G2A (FIG. 10B).

### Example 3

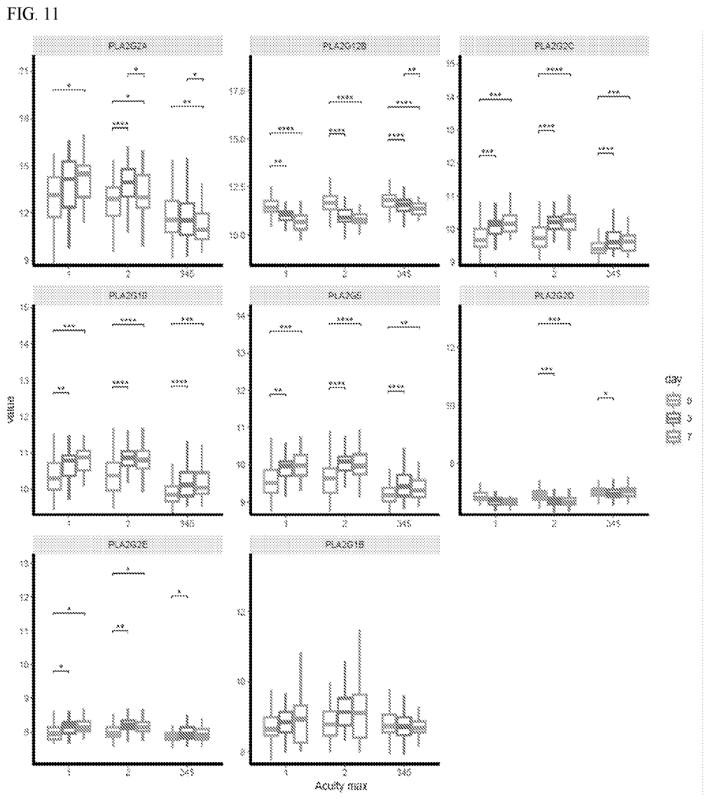

Proteomics was carried out utilizing the SomaScan Platform (4776 unique human protein targets). These data were analyzed from the 2021 study of Filbin et al. (Cell Reports Medicine 2, 100287 2021). In this study, patient blood samples were taken upon entering the hospital (day 0) and at day 3 and day 7 after hospital stay. FIG. 11 shows levels of the family of secreted PLA2s at each of the time points. Only 44 of 4776 proteins follow the kinetics of continuing to increase in the deceased patient population. This included the secreted PLA2 isoforms, PLA2G2A, PLA2G10, PLA2G5, PLA2G2C, PLA2G1B, and PLA2G2E.

All publications, patents, patent applications and accession numbers mentioned in the above specification are herein incorporated by reference in their entirety. Although the invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the following claims.

1. A method of treating a condition, disease or disorder in a subject, comprising:
a) assaying a sample from said subject for the level of a secreted phospholipase A2 (sPLA2); and
b) administering an sPLA2 inhibitor to said subject when said sample has an elevated level of sPLA2.

2. The method of claim 1, wherein said sPLA2 is a low MW, Ca⁺⁺ dependent sPLA2.

3. The method of claim 1 or 2, wherein said sPLA2 is sPLA2-IIA.

4. The method of claim 1 or 2, wherein said sPLA2 is selected from the group consisting of PLA2G12B, PLA2G1B, PLA2G16, PLA2G5, PLA2G10, PLA2G2C, PLA2G2E, PLA2G7 and PLA2G2D.

5. A method of treating a condition, disease or disorder in a subject, comprising:
a) assaying a sample from said subject for the level of secreted phospholipase A2 isoform IIA (sPLA₂-IIA); and
b) administering an sPLA2-IIA inhibitor to said subject when said sample has an elevated level of sPLA2-IIA.

6. The method of any of the preceding claims, wherein said condition, disease, or disorder is or includes multiple organ failure.

7. The method of any of the preceding claims, wherein said condition, disease or disorder is selected from the group consisting of a respiratory disorder, a trauma, a bacterial infection, septic shock, heart failure, and disseminated intravascular coagulation.

8. The method of claim 7, wherein said respiratory disorder is acute respiratory distress syndrome (ARDS).

9. The method of any of the preceding claims, wherein said subject is infected with or has been infected with the SARS-CoV-2 virus.

10. The method of claim 5, wherein said subject has one or more symptoms of COVID-19.

11. The method of claim 5, wherein said subject has PASC.

12. The method of any of the preceding claims, wherein said subject is at increased risk of severe disease or death from said condition, disease or disorder.

13. The method of any of the preceding claims, wherein said subject is over the age of 65.

14. The method of any of the preceding claims, further comprising assaying one or more of the subject's respiration rate, oxygen saturation or pulmonary lesion progression.

15. A method of treating COVID-19 in a subject, comprising:
a) assaying a sample from said subject for the level of an sPLA2; and
b) administering an sPLA2inhibitor to said subject when said sample has an elevated level of sPLA2.

16. The method of any of the preceding claims, wherein said assaying comprises an immunoassay.

17. The method of any of the preceding claims, wherein said sPLA2 inhibitor is selected from the group consisting of a nucleic acid, an antibody, and a small molecule.

18. The method of claim 17, wherein said small molecule is selected from the group consisting of varespladib methyl, AZD2716, 7,7-Dimethyleicosadienoic Acid (DEDA), oleyloxyethyl phosphorylcholine, luffariellolide, thioetheramide PC, 4-[(1-oxo-7-phenylheptyl)amino]-(4R)-octanoic acid, LY315920, and γS-[(1-oxo-7-phenylheptyl)amino]-4-(phenylmethoxy)-benzenepentanoic acid.

19. The method of claim 17, wherein said nucleic acid is selected from the group consisting of an antisense nucleic acid, a miRNA, an siRNA, and an shRNA.

20. The method of any of the preceding claims, wherein said elevated level of sPLA2 is an elevated level relative to a reference level selected from the group consisting of the level in a subject not diagnosed with a respiratory disorder, the level of said subject prior to being diagnosed with said respiratory disorder, and a population average of subjects not diagnosed with respiratory disorders.

21. The method of any of the preceding claims, wherein the sPLA2 is sPLA2-IA and said elevated level of sPLA2-IIA is above 10 ng/ml.

22. The method of claim 21, wherein said elevated level of sPLA2-IIA is above 150 ng/ml.

23. The method of any of the preceding claims, wherein said sample is blood or a blood product.

24. The method of any of the preceding claims, wherein said sPLA2 is catalytically active.

25. The method of any of the preceding claims, wherein said patient has a blood urea nitrogen (BUN) level greater than or equal to 16 mg/dl.

26. A method of treating COVID-19 in a subject, comprising:
a) assaying a sample from said subject for the level of an sPLA2 and blood urea nitrogen (BUN); and
b) administering an sPLA2 inhibitor to said subject when said sample has a level of sPLA2 above 10 ng/ml and a level of BUN above 16 ng/dl.

27. A method of treating PASC in a subject, comprising:
a) assaying a sample from said subject for the level of sPLA2; and
b) administering an sPLA2 inhibitor to said subject when said sample has a level of sPLA2 above a threshold level.

28. The method of claim 27, wherein said assaying is repeated at one or more time points.

29. The method of claim 27, wherein said assaying is repeated weekly, monthly, or yearly.

30. The method of any one of claims 27 to 29, wherein said administering is continued until said sPLA2 drops below said threshold level.

31. A method of providing a prognosis to a subject diagnosed with a respiratory disorder or multiple organ failure, comprising:
a) assaying a sample from said subject for the level of sPLA2; and
b) identifying said subject as having an increased risk of severe disease and/or death when said level of sPLA2 is elevated.

32. The method of claim 31, further comprising administering an sPLA2 inhibitor to said subject when said sample has an elevated level of sPLA2.

33. A method of providing a prognosis to a subject diagnosed with a respiratory disorder or multiple organ failure, comprising:
a) assaying a sample from said subject for the level of acetylcarnitine; and
b) identifying said subject as having an increased risk of severe disease and/or death when said level of acetylcarnitine is elevated.

34. A method of treating a condition, disease or disorder associated with mitochondrial dysfunction in a subject, comprising:
a) assaying a sample from said subject for the level of acetylcarnitine and/or mitochondrial DNA; and
b) administering an sPLA2 inhibitor to said subject when said sample has an elevated level of acetylcarnitine and/or mitochondrial DNA.

35. The use of a sPLA2 inhibitor to treat a condition, disease or disorder in a subject with an elevated level of sPLA2.

36. An sPLA2 inhibitor for use in treating a condition, disease or disorder in a subject with an elevated level of sPLA2.

37. The use of a sPLA2 inhibitor to treat a condition, disease or disorder in a subject with an elevated level of acetylcarnitine and/or mitochondrial DNA.

38. An sPLA2 inhibitor for use in treating a condition, disease or disorder in a subject with an elevated level of acetylcarnitine and/or mitochondrial DNA.
